(19) United States
(12) Patent Application Publication
(10) Pub. No.: US 2015/0073008 A1
(43) Pub. Date: Mar. 12, 2015

(54) Topical Application of Vinca Alkaloids for the Treatment of Actinic Keratosis

(71) Applicant: Merz Pharma GmbH & Co. KGaA, Frankfurt am Main (DE)

(72) Inventors: Bhushan Hardas, Summerfield, NC (US); Harry Frank Abts, Oberursel (DE); Thomas Hengl, Frankfurt (DE); Kim Schlinzig, Oberursel (DE); Alan Fleischer, Winston-Salem, NC (US); Sarah Caley, Chapel Hill, NC (US); Jon Parrish, Greensboro, NC (US); Lutz Franke, Bad Vilbel (DE)

(21) Appl. No.: 14/483,823

(22) Filed: Sep. 11, 2014

(30) Foreign Application Priority Data
Sep. 12, 2013 (EP) 13184113.2

(51) Int. Cl. A61K 31/439; A61K 9/00

(52) U.S. Cl. CPC A61K 31/439; A61K 9/0014
USPC 514/283

(57) ABSTRACT

The present invention provides pharmaceutical compositions comprising a vinca alkaloid of formula (I):

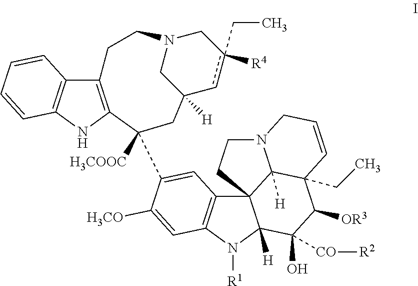

or a pharmaceutically acceptable salt (or hydrate) thereof, wherein each of R¹, R², R³, and R⁴ is as defined and described herein, wherein said formulations are useful for the topical treatment of actinic keratosis.

### CROSS-REFERENCE TO RELATED APPLICATIONS

The present application claims priority to European patent application 13184113.2, filed Sep. 12, 2013, the entirety of which is hereby incorporated herein by reference.

### TECHNICAL FIELD OF THE INVENTION

The present invention relates to pharmaceutical formulations of vinca alkaloids for the treatment of actinic keratosis and to a method for identification of compounds targeting proliferating epidermal cells and the use of such compounds for the treatment of actinic keratosis. The present invention also relates to topical formulations comprising a vinca alkaloid and the uses thereof for the treatment of clinical conditions of body surfaces, in particular of skin, wherein abnormal cell differentiation and/or hyper-proliferation is a primary factor of the pathogenesis.

### BACKGROUND OF THE INVENTION

In the last years, a particular type of skin-cell dysfunction has become more of interest, the disease called actinic keratosis. In general, actinic keratosis (also called “solar keratosis” and “senile keratosis”) is a pre- or early-malignant condition of thick, scaly or crusty patches of skin consisting of dysplastic keratinocytic lesions. Actinic keratosis (AK) has become a common condition treated by dermatologists. AK is associated with persons (or animals) frequently exposed to the sun, as it is usually accompanied by solar damage. These lesions can progress to squamous cell carcinoma (SCC) Annual rates of transformation are often cited from 0.1%-20% and thus these pre-cancerous lesions should be treated early.

The lesions related with actinic keratosis can be treated to provide relief from early symptoms, such as tenderness or itch. When skin is exposed to the sun constantly, thick, scaly, or crusty bumps may appear. The scaly or crusty part of the bump is dry and rough. The growths start out as flat scaly areas, and later grow into a tough, wart-like area. In addition to chronic UV-exposure also infections with viruses, such as HPV, has been implicated in the etiology of actinic keratosis. An actinic keratosis site often ranges from 2 to 8 mm in size and can be a dark or light, tan, pink, red spot, or a combination of these, or have the same pigment as the surrounding skin. It typically appears on any sun-exposed area of the skin, such as the face, ears, neck, scalp, chest, backs of hands, forearms and/or lips.

Different types of medical treatments for actinic keratosis have been developed but they have some inconveniences related to the treatment conditions and/or side effects. Thus, there remains an unmet need to develop treatments for actinic keratosis and related disorders.

### BRIEF DESCRIPTION OF THE DRAWINGS

FIG. 1 depicts the DNA-content (%) as a function of Vindesine concentration (nM) for 20% FBS medium cultivated cells.

FIG. 2 depicts the DNA-content (%) as a function of Vindesine concentration (nM) for 0% FBS medium cultivated cells.

### DETAILED DESCRIPTION OF CERTAIN EMBODIMENTS OF THE INVENTION

In certain embodiments, the present invention relates to pharmaceutical formulations of vinca alkaloids for the treatment of actinic keratosis and to a method for identification of compounds targeting proliferating epidermal cells and the use of such compounds for the treatment of actinic keratosis. In certain embodiments, the present invention also relates to topical formulations comprising a vinca alkaloid and the uses thereof for the treatment of clinical conditions of body surfaces, in particular of skin, wherein abnormal cell differentiation and/or hyper-proliferation is a primary factor of the pathogenesis.

Several vinca alkaloids and their preparation from natural sources are known for many decades. The vinca alkaloids vinblastine and vincristine were originally found in the Madagascar periwinkle (Vinca rosea). Vindesine and vinorelbine are semisynthetic derivatives of vinblastine. The natural and semisynthetic derivatives of vincristine are collectively referred to herein as vinca alkaloids. These vinca alkaloids act by inhibiting mitosis in metaphase and bind to tubulin, thus preventing the cell from making spindles. The vinca alkaloids interfere indirectly with the ability of cells to synthesize DNA and RNA.

For therapeutic uses, vinca alkaloids are currently administered systemically by injection. Systemic administration of vinca alkaloids however can result in a number of undesirable side effects. For the treatment of lymphoma, vinca alkaloids are administered intravenously in their sulfate form, often once a week. Several naturally alkaloids obtainable from Vinca rosea are known for years for the treatment of leukemia. From U.S. Pat. No. 3,097,137 the compound vincaleukoblastine and from U.S. Pat. No. 4,479,957 the compound vindesine for treatment of lymphatic leukemia, lymphoma, malignant melanoma and breast cancer are known.

Sterile injection compositions of vincristine, vinblastine and vindesine are known from U.S. Pat. No. 4,619,935. The documents WO 1989/11292, WO 2000/59473 and US 2004/0071768 present liposome-encapsulated vinca alkaloids for intravenous treatment of lymphomas. From WO 2001/64210 a method for treating neoplasia is known using antineoplastic vinca alkaloid derivatives (U.S. Pat. No. 4,307,100) together with phosphodiesterase inhibitors (PDE) in order to reduce the side effects of vinca alkaloid derivative treatment. The combination is applied in in-vitro tests against neuroplasia.

Further liposome-encapsulated formulations of vinca-derivatives are described in WO 2005/011698 and WO 2005/107712. In WO 2003/015759, anthracyclines, such as valrubicin, are described which can be used for the treatment of neoplastic processes and hyper-proliferation, such as psoriasis, carcinoma of the skin and actinic keratosis. A method for controlling aberrant cell proliferation is described in WO 2005/027907, which comprises contacting the cells with a check point kinase (Chk-1) activator, such as a chemotherapeutic agent, and then contacting the cells with a (Chk-1) inhibitor.

The document WO 2008/064425 describes a method for systemic treating tumorous growth by administering a combination of glyco-alkaloids with mitotic inhibitors, such as vinca alkaloids. From WO 2012/154942 some ceramide-liposome-formulations for all types of applications comprising a vinca-alkaloid, such as vinblastine are known. WO 2012/054923 discloses polymeric nanoparticles that can include e.g. a vinca-alkaloid as therapeutic agent, for infusion or injection. Some release data from the formulations are provided.

In the publication of M. C. Massa (Angiolymphoid hyperplasia demonstrating extensive skin and mucosal lesions controlled with vinblastine therapy, Journal of Amer. Academy of Dermatology, Vol. 11, No. 2, p. 333-339, 1. August 1984) the intravenous treatment of angiolymphoid hyperplasia with vinblastin sulfate is described.

In EP-A 0 041 030, a mixture of alkaloids obtained as extract from the plant Vinca rosea is described e.g. for a local treatment of hyper-seborrhoeic conditions targeting specifically the hair-follicle. However, the alkaloids mentioned (e.g. raubasine and bromocriptine) have different chemical structures and different pharmacological targets compared to vinca alkaloids of the present invention.

The compound vinblastine is mainly useful for treating Hodgkins disease, lymphocytic lymphoma, advanced testicular and breast cancer and Kaposis sarcoma. Common side effects of systemic use include hair loss, nausea, lowered blood cell counts, headache and stomach pain. Vincristine is used for years mainly for i.v. treatment of acute leukemia, sarcoma, neuroblastoma, Hodgkins disease and other lymphomas. Side effects include those found with vinblastine, plus additional nervous system problems such as sensory impairment.

Vindesine is used to treat melanoma, lung and breast cancer and, in combination with other drugs, to treat uterine cancers. It is often administered at a dose of 3 milligrams (i.v.) per square meter of body surface. Vinorelbine is indicated for breast cancer and the first-line treatment of non-small cell lung cancer (NSCLC), sometimes in combination with other drugs, e.g. cisplatin.

Therefore, it is one aspect of the present invention to provide a topical treatment with specific vinca alkaloids and pharmaceutical formulations for use for an efficient treatment of actinic keratosis with tolerable side effects. These formulations are useful to treat larger areas of affected skin (“field treatment” in addition to “spot treatment”) and to eliminate obvious actinic keratosis lesions as well as clinically non visible pre-lesions. This is beneficial for many patients and a major improvement over existing, very limited actinic keratosis therapies.

The currently widely used procedures that are used to treat actinic keratosis, such as cryosurgery, require technical skill, are painful and produce erythema, blistering, and/or a prolonged healing process. Cryosurgery requires ready access to a cryogen, such as liquid nitrogen, and specialized equipment suitable for delivery of the cryogen. Current topical treatments of actinic keratosis also have the potential of being very unsightly and painful. These drug treatments include the following.

Treatment with the known drug 5-fluorouracil (5-FU) as a very aggressive substance is often used in cancer therapy.

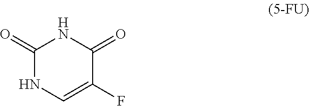

This compound works through noncompetitive inhibition of thymidylate synthase. Due to its non-competitive nature and effects on thymidine synthesis, 5-FU is referred to as the “suicide inactivator”. It is an S-phase specific drug and thus preferentially effecting stronger proliferating cells. Due to its general impact on thymidine-metabolism it has an impact also on metabolic active non-dividing cells. Common local application related side effects of 5-FU include pain, burning, itching, redness, crusting, oozing and bleeding. Additionally, topical application may cause abdominal pain, bloody diarrhea, vomiting, fever, stomatitis, and inflammation. Systemically administered 5-FU also causes acute CNS damage.

The treatment with Imiquimod (Mw 240 g/mol; Aldara or Zyclara) activates immune cells through the toll-like receptor 7 (TLR7), commonly involved in pathogen recognition on the cell surface. Cells activated by Imiquimod via TLR-7 secrete cytokines, primarily interferon-α (IFN-α), interleukin-6 (IL-6) and tumor necrosis factor-α (TNF-α). Imiquimod, when applied to skin, can lead to the activation of Langerhans cells, which migrate to local lymph nodes to activate the adaptive immune system. Other cell types activated by Imiquimod include natural killer cells, macrophages and B-lymphocytes. Common local application-related side effects include pain, burning, itching, irritation and bleeding. Systemic influenza-like symptoms may occur with topical application.

The drug Diclofenac (Mw 296 g/mol; DF, Solaraze) is a long known non-steroidal anti-inflammatory drug (see U.S. Pat. No. 3,558,690), which leads to an increased apoptosis.

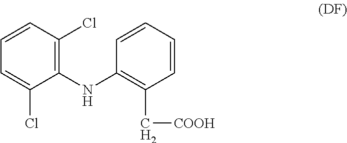 [SMILES: O=C(O)Cc1ccccc1Nc1c(Cl)cccc1Cl]

The efficacy of DF is substantially less than Imiquimod and 5-FU. The treatment periods can be as long as 90 days, which is a burden for patients. Reported side effects include contact dermatitis, rash, dry skin and scaling.

The photodynamic therapy (PDT) for actinic keratosis uses photosensitive drugs (such as 5-ALA and Metvix), which are triggered by light of a specific wavelength, usually red or infrared. PDT is only suitable for lesions (“spots”) rather than for a field treatment (e.g. more than 1% of skin surface). When the drug is applied, a waiting period is required, followed by a light exposure requiring specialized equipment. Reported local side effects include scaling, pain, tenderness, itching, edema, ulceration, bleeding and erosion.

5-FU and Imiquimod are very uncomfortable and unsightly treatments, especially for the milder forms of actinic keratosis. Therefore, there is a particular need for a gentler treatment of AK which has reduced side effects and can also be applied to larger areas of the skin.

The skin of an adult normally covers an area of about 1.5 to 2 m². Often more than 0.05 percent, in particular 0.1 to 10 percent, often 0.5 to 10 or 0.5 to 9 percent of the skin (surface) needs to be treated, which requests particular attention with respect to the formulation. Also, the active product (API) has to have a good tolerability. The vinca alkaloid formulation should be easy to administer, also to larger areas of the skin, to allow a field treatment (e.g. more than 1% of skin surface). For some known API, such as Imiquimod, such field treatment is difficult.

Furthermore, the stability of the formulations (compositions comprising a vinca alkaloid) over a longer period of time should be given, in particular at room temperature (20° C.), or in the range of 4 to 30° C., in particular 20 to 25° C. Also the stability at neutral pH-values, at about 7 to 7.4, as well as at pH-values below 7, such as pH 5.5 to 3.0 is of particular interest.

In certain embodiments, the present invention therefore relates to new pharmaceutical compositions for topical application for improving the appearance of (damaged, in particular sun-damaged) skin, in particular for treating actinic keratosis, comprising at least one vinca alkaloid, or a pharmaceutically acceptable salt thereof. The composition preferably comprises at least one dermatologically acceptable excipient. The vinca alkaloid can be chosen from a large group of compounds. In certain embodiments, the vinca alkaloid is selected from vindesine, vinblastine, vincristine and vinorelbine. Each of these vinca-compounds (and their salts) was found to have particular useful properties, such as skin permeability, stability and cutaneous tolerance. These four vinca alkaloids (and the salts or hydrates thereof) are often used with a high degree of purity, such as >95%, often >99%.

Interestingly, the further known drug compounds teniposide (Mw 666 g/mol; U.S. Pat. No. 3,524,844) and epirubicin (Mw 544 g/mol; U.S. Pat. No. 4,058,519), which completely differ in structure from the vinca alkaloids, also can be used for provided topical pharmaceutical formulations and methods using the same. They were found by the specific screening method to show good DCF-values, as described in detail in the Examples, herein.

In certain embodiments, the vinca alkaloids can be in the form of pharmaceutically acceptable salts. In certain embodiments, the vinca alkaloids can also be in the form of hydrates, such as sulfate hydrates. Pharmaceutically acceptable salts refer to those salts which possess the biological effectiveness and properties of the parent compound and which are not biologically or otherwise undesirable. These salts (but also the vinca compounds per se) often comprise specific amounts of water, in some cases they form hydrates.

Typical examples of acids which can be used for salt formation of provided vinca alkaloids are:

Acetic acid, N-acetylglycine, Adipic acid, Alginic acid, L-Ascorbic acid, L-Aspartic acid, Benzenesulfonic acid, Benzoic acid, Benzoic acid, 4-acetamido-, Benzoic acid, 2-(4-hydroxybenzoyl)-Butyric acid, (+)-Camphoric acid (+)-Camphor-10-sul-fonic acid, Caproic acid, Caprylic acid, Carbonic acid, Cinnamic acid, Citric acid, Cyclamic acid, Decanoic acid, Di(tert-butyl)naphthalenedisulfonic acid, Di(tert-butyl)naph-thalenesulfonic acid, Dodecylsulfuric acid, Embonic acid (see pamoic acid), Ethane-1,2-disulfonic acid, Ethanesulfonic acid, Ethanesulfonic acid, 2-Hydroxy-formic acid, Fumaric acid, Galactaric acid, Gentisic acid, D-Glucaric acid, D-Glucoheptonic acid, D-Gluconic acid, D-Glucuronic acid, L-Glutamic acid, Glutaric acid, Glutaric acid, -oxo-, Glycerophosphoric acid, Glycolic acid, Hexanoic acid, Hippuric acid, Hydrobromic acid, Hydrochloric acid, Isobutyric acid, Isethionic acid, Ketoglutaric acid, DL-Lactic acid, Lactobionic acid, Lauric acid, Maleic acid, (−)-L-Malic acid, Malonic acid, DL-Mandelic acid, Methanesulfonic acid, Mucic acid, Naphthalene-1,5-disulfonic acid, Naphthalene-2-sulfonic acid, 2-Naphthoic acid, 1-hydroxy-, Nicotinic acid, Nitric Acid, Octadecanoic acid, Octanoic acid, Oleic acid, Orotic acid, Oxalic acid, Palmitic acid, Pamoic acid, Phosphoric acid, Propanoic acid, (−)-L-Pyroglutamic acid, Pyruvic acid, Saccharin, Salicylic acid, Salicylic acid, 4-amino-, Sebacic acid, Stearic acid, Succinic acid, Sulfuric acid, (+)-L-Tartaric acid, Thiocyanic acid, para-Toluenesulfonic acid, Trifluoroacetic acid, and Undec-10-enoic acid.

In certain embodiments, the salts of vindesine, vinblastine, vincristine and vinorelbine are the sulfates, di-tartrates, hydrochlorides, lactate, maleate, 1,5-naphthalenedisulfonates and trifluoroacetates. In certain embodiments, the salts of vinca alkaloids are selected from:

Vinblastine: sulfate, hydrochloride, naphthalene disulfonate and trifluoroacetate;

Vincristine: sulfate and tartrate;

Vindesine: sulfate, tartrate and 1,5-naphthalene disulfonate;

Vinorelbine: ditartrate, sulfate, lactate and, malate.

For the pharmaceutical application, the vinca alkaloids, either alone or preferably together with one or more excipients, are placed into the form of pharmaceutical composition or formulation (and unit dosages thereof) and in such form are employed, in particular for topical use, on the skin. Such pharmaceutical formulations may comprise conventional or new ingredients, with or without additional active substances. Preferably, the vinca alkaloids are used without further pharmaceutically active compounds in the formulation. The pharmaceutical formulations may contain any suitable effective amount of the vinca alkaloid (or its salt or hydrate) commensurate with the intended daily (or weekly) dose regiment to be employed.

Due to their high degree of activity against hyper-proliferating cells, the vinca alkaloids can be administered to a subject, a living animal or a human body, in need thereof, for the treatment, alleviation, or amelioration, palliation, or elimination of actinic keratosis. The compositions comprising the vinca alkaloid (or its salt or hydrate) are preferably formulated together with one or more pharmaceutically (or dermatologically) acceptable excipients. These compositions are especially in the form of topical pharmaceutical formulations.

The present invention discloses several vinca alkaloids as well as derivatives (in particular salts and hydrates) thereof, which may be used in topical formulations (pharmaceutical compositions) for the treatment of actinic keratosis and other clinical conditions of body surfaces, wherein hyper-proliferation (of skin associated cells) and/or ectopic-proliferation is a primary factor of the pathogenesis. It is an objective of the present invention to provide pharmaceutical compositions comprising at least one vinca derivative together with one or more pharmaceutically acceptable excipient wherein said pharmaceutical composition is formulated for topical administration to a body surface, in particular skin of head, arms, legs and other parts frequently exposed to sun light. The compositions are specifically for the treatment of unwanted or ectopic proliferating skin-cells.

It is a further objective of the present invention to provide methods for treatment of a condition or disease associated with hyper-proliferation or pre-neoplastic or neoplastic processes of a body surface, in particular of actinic keratosis, in an individual in need thereof, comprising administering a pharmaceutical composition, comprising at least one vinca alkaloid (or its salt or hydrate), topically to said individual.

It is a further objective of the present invention to provide kits for pharmaceutical compositions comprising a vinca alkaloid (or its salt or hydrate), for the preparation of a medicament (medication) for the treatment of a condition associated with hyper-proliferation of a body surface, in particular for actinic keratosis.

The pharmaceutical composition of the present invention is preferably for the treatment of proliferating skin-cells and in particular for the treatment of actinic keratosis.

In certain embodiments, a pharmaceutical composition of the invention preferably comprises a vinca alkaloid of formula (I):

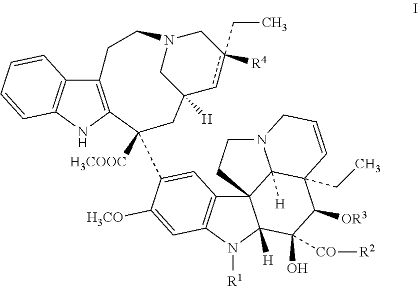

or a pharmaceutically acceptable salt (or hydrate) thereof, wherein:
R¹ is C₁-C₃-alkyl, C₁-C₃-alkoxy, or —CHO;
R² is C₁-C₃-alkyl, C₁-C₃-alkoxy, or amino;
R³ is CO—CH₃, methyl, or hydrogen; and
R⁴ is hydroxyl or hydrogen.

In some embodiments, R¹ is methyl or CHO.

In some embodiments, R² is methoxy or amino.

In some embodiments, R⁴ is hydroxyl.

In particular the radicals in formula (I) denote: R¹ methyl or —CHO; R² methoxy or amino and R³ is CO—CH₃ or hydrogen. R⁴ denotes hydroxyl or hydrogen, often it denotes —OH. The dotted line in formula (I) in the ring system, carrying R⁴, shall indicate that this ring can be saturated or can contain a double bond, such as in the drug compound vinorelbine. One of ordinary skill in the art will recognize if this ring contains a double bond, R⁴ is absent. In some embodiments, this ring does not contain a double bond.

In some embodiments, a providedpharmaceutical composition preferably comprises at least one vinca alkaloid from the group consisting of vindesine, vinblastine, vincristine and vinorelbine (or pharmaceutically acceptable salts (or hydrates) thereof). However, other vinca alkaloids, e.g. vindoline, vinleurosine, vinrosidine, can also be used.

Suitable dosage ranges, in particular of the vinca alkaloids of formula (I), are e.g. from 0.001 to 500 milligrams daily, sometimes from 0.01 to 250 milligrams daily, often from 0.1 to 150 milligrams daily, preferably 0.2 to 100 milligrams daily, depending upon the exact formulation, the stage of disease toward which the administration is directed, the subject involved and the body weight (and body surface) of the subject involved, and the preference and experience of the physician or veterinarian in charge.

Suitable concentrations of vinca compounds in the pharmaceutical compositions, in particular of the vinca alkaloids of formula (I), are from 0.0001 to 10% by weight, such as 0.001 to 10% by weight, often however from 0.001 to 0.01% by weight, in particular 0.002 to 0.008% by weight, such as 0.005% by weight, or in some embodiments from 0.05 to 5% by weight, of the final composition to be applied. In some embodiments of the invention the vinca alkaloids of formula (I), are applied in a concentration from 0.01 to 10% by weight, of the final composition. These concentrations also apply to the group of vindesine, vinblastine, vincristine, vinorelbine and pharmaceutically acceptable salts or hydrates thereof

The amount of respective vinca alkaloid to be found in the epidermis (after topical administration of the composition) often corresponds to the 1-500 fold, often 10-100 fold, in particular 10-50 fold in vitro IC50-value (e.g. in SCC-111 cells) of the vinca alkaloid.

Whereas vinorelbine (ditartrate) has e.g. a low in vitro IC50-value of 1.34 nano-mol (in SCC-111 cells), the compound 5-FU has an IC50-value of 960 nano-mol in this test system. Vindesine (sulfate) has e.g. an IC50-value of 0.57 nano-mol, Vinblastine (sulfate) has e.g. an IC50-value of 0.87 nano-mol, Vincristine (sulfate) has e.g. an IC50-value of 0.92 nano-mol.

In particular the topical formulations of vindesine, vinblastine, vincristine and vinorelbine and the pharmaceutically acceptable salts (and hydrates) thereof, are well tolerated by the skin and undesired irritant effects can be avoided. In vitro studies with human skin (Leiden epidermal skin) and with the four vinca compounds (vindesine, vinblastine, vincristine and vinorelbine) showed in the Methylthiazol Tetrazolium-Assay (MTT) that even at higher doses (of 100-times the in vitro 1050-values) these vinca-alkaloids were not rated as irritant to the skin (e.g. metabolic activity of skin model not reduced to ≦50%). This leads to the option to apply the vinca-compositions not only to single affected skin lesions (“spots”) but also to larger areas of the skin, e.g. for “field treatment”, and often even >5% (or >10%) of the skin surface (of the individual).

Whereas the classical treatment with 5-FU was found to kill not only proliferating skin cells but generally to drastically reduce also the number of resting (i.e. non-proliferating) cells, the vinca alkaloids show a clean cytostatic effect by selectively targeting only proliferating cells. Flow-cytometric analysis identified a high-rate of apoptosis in cells treated with vinca alkaloids according to this invention.

Thus, with topical formulations of the suggested vinca alkaloids, the side effects of general cytotoxicity can be avoided in the skin. This was also shown in comparative tests by measuring the cell density as a function of time for 5-FU (comparison) and the vinca alkaloids.

By application of vinca alkaloids, the number of cells in samples with normal (i.e. proliferating) and high cell density (i.e. non-proliferating) was kept constant due to targeting only proliferating cells, with 5-FU cells were affected, independent of their proliferation-status. Thus, cells were killed within a short period of time, at normal (proliferation) and high cell density (no-proliferation). The high amount of apoptosis found to be induced by the vinca alkaloids of the present invention offers upside therapeutic advantages, e.g. as apoptosis does not lead to similar inflammation as necrotic cell death.

While the selective activity of the claimed vinca alkaloids allows spot-treatment of individual lesions, due to their high dermal tolerability and their ability to induce apoptosis, the topical formulations are furthermore applicable to larger areas of the skin, allowing to treat typical sun-exposed areas at once (e.g. head, neck, face, arms).

Thus, with the claimed vinca alkaloids and their topical formulations, the advantageous treatment of the whole (or significant parts of) sun affected regions is possible. Most commonly, the face, scalp, neck, hands and forearms have been identified as regions in need of considering field treatment.

Typically skin areas between 0.1 to 60%, often 0.1 to 20% (of the total skin surface) can be treated. Often, 0.1 to 10%, often 0.5 to 10% or 0.5 to 9% of the skin surface of the individual is treated (at once). Field treatment can involve more than 0.5 percent, in particular more than 1 percent of the skin surface, or even more than 2.5% of the skin surface. It is possible to also treat skin areas neighboring the specific lesions (spots) or to treat areas which do not (yet) show lesions.

The topical formulations preferably also have a long-term stability at an elevated temperature of 37° C. and a pH-value below 6, such as a pH-value of 5.5, of more than one month, in particular more than 3 months. In one embodiment, the topical formulations of vindesine, vinblastine, vincristine and vinorelbine have a long-term stability at 37° C. of more than 12 month (>90% compound detected).

Some topical formulations may have a reduced stability at a temperature of 37° C. and a pH-value of 7 or more, e.g. a pH-value of 7.4. For the preferred group the following stability results were found at pH 5.5 (test for 7 days, 5° C.): vindesine sulfate 98.8%, vinblastine sulfate 97%, vincristine sulfate 99.6% and vinorelbine ditartrate 99.5%.

The topical formulations of the vinca alkaloids preferably have a sufficient level of dermal absorption, which allows a significant amount of the vinca alkaloid to reach the stratum corneum, and in particular the epidermis. This was shown by in-vitro testing of vinca formulations (0.001 mg vinca compound/cm²) with human skin probes (see Dermal Absorption Studies with human skin). As one example, for a vinorelbine ditartrate crème formulation, a penetration of more than 0.7% of the vinca compound in the epidermis was found.

The vinca alkaloids were tested in different concentrations and in different formulations (such as cream, liposomes and hydro-tops). The vinca alkaloids, in particular vindesine, vinblastine, vincristine, vinorelbine and pharmaceutically acceptable salts thereof, showed a good dermal absorption. Furthermore, based on in vitro dermal absorption studies, they were found to remain mainly in the skin and not to become available to a substantial level systemically. It is important, that the amount of the vinca alkaloid is immediately delivered to the subcutaneous layers and/or the amount in the blood system is kept very low (e.g. less than 1% of the active compound).

Therefore, preferred vinca alkaloids according to the present invention are chosen so that they are systemically absorbed in a limited manner, after topically administration to the body surface, in particular the skin. Preferably, the immediate systemic absorption is less than 10%, such as less than 5%, for example less than 1%. The systemic absorption can be determined by conventional methods, for example by measuring the amount of vinca alkaloid in a blood sample or in serum by HPLC.

Preferred vinca alkaloids according to the present invention display locally not a general toxicity, when applied topically to the skin of an individual. Preferably, the vinca alkaloids are not or are only mildly irritant when applied to the body surface, such as skin, in an effective dose. The body surface may be skin or mucosal membranes but normally it is the skin of a person. Skin is composed of epidermis and dermis.

The epidermis usually comprises several (e. g. five) layers, which from inside to outside are stratum basale, stratum spinosum, stratum granulosum, stratum lucidum and stratum corneum, wherein the stratum corneum consists of dead, keratinised cells (corneocytes) embedded in a lipid matrix. Each of these layers may comprise one or more cell layers. The dermis underlies the epidermis and is a dense irregular connective tissue. The dermis may furthermore for example comprise hair follicles, sweat glands and/or nerve endings. The term “skin” within the present invention can comprise one or more or the above mentioned layers and structures.

The conditions and diseases to be treated according to the present invention are conditions of a body surface, in particular skin, associated with hyper-proliferation and/or pre-neoplastic and/or neoplastic processes. Preferably, the condition is a condition, wherein hyper-proliferation or ectopic proliferation is a primary factor of pathogenesis.

In one preferred embodiment of the present invention, the condition is associated with dermal hyper-proliferation, which may for example be hyper-proliferation of epidermis or hyper-proliferation of dermis. Preferably hyper-proliferation of the epidermis is treated by using topical formulations of vinca alkaloids.

For example, the condition according to the present invention may be selected from the group consisting of actinic keratosis, seborrheic keratosis and photo-induced keratosis.

In one preferred embodiment the condition is actinic keratosis, where this term includes all types of actinic keratosis known to the person skilled in the art. The keratosis to be treated may be mild, moderate, more severe or very severe. The formulations in one embodiment can be applied to patients, where (in a spot treatment), less than 0.01 percent, often less than 0.001 percent of the skin is affected, but also larger area of the skin can be treated. In one preferred embodiment of the present invention, for spot treatment 0.0005% to 0.005%, such as 0.001% to 0.003%, like 0.002%, of the total body surface area is treated.

The topical formulations of the vinca alkaloid can be used for ameliorating treatment and/or for curative treatment and/or for prophylactic treatment. The treatment may abolish or relieve some or all of the symptoms of actinic keratosis during treatment and/or for a specific period of time after cessation of treatment.

The pharmaceutical formulations according to the present invention are preferably administered topically, which can be understood as local administration directly to the site of disease, normally the skin. Preferably, the topical administration results in that the majority of the vinca alkaloid is not (or little) systemically absorbed and substantially capable of exerting its effect locally at the site of application. The administration of the vinca formulation may be only once, however, the treatment is usually administered more than once, such as daily for 10 to 40 days (or longer). In some embodiments of the invention, the vinca alkaloids are administered up to 90 days. In some embodiments of the invention shorter treatment durations such as 2 to 7 days are applicable.

In a preferred embodiment of the present invention, the individual administrations are distributed over a period of 7 to 30 days or 10 to 30 days. Furthermore, the time gap between two individual administrations may be less than 1 day, for example 12 hours, but also can be e.g. one day or more, such as 2 days. Alternatively, the time gap between two individual administrations can be 3 to 7 days, e.g. 4 to 5 days. The time gap between two individual administrations may always be the same or it may differ from time to time.

The dose to be administered depends on the particular individual and condition and severity of the actinic keratosis to be treated as well as the specific formulation for the administration of the vinca alkaloid. For the topical administration, a topical composition, comprising the vinca alkaloid and the excipient(s) can in particular be an ointment, lotion, cream, foam, solution or gel in particular aspects.

Topical dosage forms such as ointment, lotion, cream foam, solution or gel bases are described in Remington: The Science and Practice of Pharmacy, 21st Ed., Lippincott Williams & Wilkins, 2006, p. 880-882 and p. 886-888; and in Allen, L. V., Ansel's Pharmaceutical Dosage Forms and Drug Delivery Systems, 8th Ed., Lippincott Williams & Wilkins, 2005, p. 277-297.

The term “excipient” applied to pharmaceutical compositions refers to a diluent, adjuvant or carrier with which the active compound, in particular of formula (I) is administered. Such pharmaceutical excipients often are sterile liquids, such as water or saline preparations. Other excipients, depending on the type of administration, can be oils, including those of animal, vegetable or synthetic origin (see A. R. Gennaro, 20th Edition of Remington, 2003: The Science and Practice of Pharmacy”). The pharmaceutical compositions according to the present invention are preferably formulated in a manner suitable for topical administration to a body surface, in particular the skin.

The pharmaceutical composition may be formulated in different ways dependent on the individual to be treated and the site of treatment. Preferably, the pharmaceutical composition may be selected from the group consisting of a lotion, an ointment, a gel, a cream, foam, solution, or a cream-gel. In particular, for vindesine, vinblastine, vincristine, vinorelbine and pharmaceutically acceptable salts (or hydrates) thereof, compositions comprising 1 to 30%, often 15 to 25% by weight of ethanol are of particular interest. Furthermore, aqueous formulations are of interest. The vinca compositions with a neutral pH-values, at about 7 to 7.4, as well as at pH-values below 7, such as pH 5 to 3, in particular from 3 to 5.5, e.g. from 4.5 to 5.5 or from 5.0 to 6.0, such as 5.5, are of particular interest.

This formulation comprises a solution and/or suspension comprising the vinca alkaloid or salts thereof. Pharmaceutically acceptable salts of the compounds according to the present invention are prepared in a standard manner. As the parent compounds are often a base, they are treated with an excess of an organic or inorganic acid in a suitable solvent.

For the vinca alkaloids and in particular for the group consisting of vindesine, vinblastine, vincristine and vinorelbine and pharmaceutically acceptable salts thereof, the following formulations are of particular interest:

Lotions, creams, ointments, gels, foams, and cream-gels according to the present invention are semi-solid formulations of the active ingredient for external application. They may be made by mixing the vinca alkaloid in finely divided or powdered form, alone or in solution or suspension in an aqueous or non-aqueous fluid, with the aid of suitable machinery, with a greasy or non-greasy base.

Examples of excipients are bases that may comprise one or more hydrocarbons such as hard, soft or liquid paraffin, glycerol, paraffin oil, beeswax, a metallic soap; an oil of natural origin such as almond, corn, arachis, castor or olive oil or derivatives thereof; wool fat or its derivatives or a fatty acid and/or ester such as steric or oleic acid. Also medium-chain fatty acid triglycerides and isopropyl myristate are of interest.

The excipient may furthermore be an alcohol such as propylene glycol, poly-ethyl ene glycol (PEG) of different molecular weights, cetyl alcohol, ethanol or a macro gel, where cetyl alcohol and ethanol are of particular interest. The pharmaceutical composition may as excipient incorporate any suitable surface active agent or emulsifier such as an anionic, cationic or non-ionic surfactant such as a sorbitan ester, polysorbate, Cremophor EL, Tween 20 or a polyoxyethylene derivative thereof. Suspending agents such as natural gums, cellulose derivatives or inorganic materials such as silicas, and other ingredients such as lanolin, may also be used as excipients.

Lotions according to the present invention include those normally suitable for application to the skin. The lotions may also include an agent to hasten drying and to cool the skin, such as an alcohol or acetone, and/or a moisturizer, such as glycerol or an oil such as castor oil.

In one embodiment, the pharmaceutical formulations according to the present invention comprise one or more compounds selected from the group consisting of emulsifiers, alcohols (e.g. as permeation enhancer) and lipids.

The emulsifier may be any emulsifier known to the person skilled in the art that is suitable for pharmaceutical formulations for topical administration. The emulsifier can e.g. be selected from the group consisting of Cremophor EL, Tween 20, polysorbate 80, Macrogol-20-glycerolmonostearat and mixtures thereof.

The alcohol compound as excipient can e.g. be selected from the group consisting of ethanol, glycerol, propylene glycol, polyethylene glycol (PEG), cetyl alcohol and mixtures thereof. PEG may be any molecular weight PEG, preferably however PEG 6000.

The lipid as excipient may be any lipid known to the person skilled in the art that is suitable for pharmaceutical formulations for topical administration.

The term lipid comprises fatty acids and esters thereof. Preferably, the lipid is selected from the group consisting of fatty alcohols, fatty acid esters, mineral oil, oil of natural origin and derivatives thereof and mixtures thereof.

Examples of specific formulations according to the present invention are given in the examples herein below, in particular for the vinca alkaloids of the group consisting of vindesine, vinblastine, vincristine and vinorelbine and pharmaceutically acceptable salts thereof

The pharmaceutical composition can be provided for the once daily application for the treatment of actinic keratosis, for a period of 10 to 40 days, often 10 to 30 days, preferably 5 to 30 days.

The invention also relates to a process for the preparation of a pharmaceutical composition for the topical application on damaged skin comprising at least one vinca alkaloid or a pharmaceutically acceptable salt thereof and comprising at least one dermatologically acceptable excipient, comprising the step of mixing the components.

A further aspect of the invention relates to a kit of parts comprising a device for the separated storing and mixing of a vinca alkaloid with the excipient(s), comprising a container which has an open end and a closed end, and comprising a cap which is arranged at the open end of the container, where the container has two or three (or more) chambers which are separated from each other by a wall, wherein the wall extends from the closed end to the open end of the container, and where at least one projecting element is constructed in the cap in such a manner that the vinca alkaloid from one of the chambers can be mixed by the projecting element with the excipients of the other chamber of the container. In the three-chamber-variant the freeze-dried vinca-alkaloid is first solubilized with an appropriate solvent by combining the content of the first and second chambers.

Finally the solubilized vinca alkaloid is mixed with the base formulation in the third chamber. To aid homogenous mixing, the system may contain a colour-indicator for indication of sufficient mixing of the different components.

As many types of drug substances have already been tested for treating proliferating epidermal cells, it is also on task of the invention to provide a better way of screening for active compounds. This method should be easy to apply and provide with reliable results for the development of drug compounds for the treatment of the respective dysfunctions or diseases. Therefore, a further aspect of the invention relates to a method for identifying compounds for the targeting of proliferating epidermal cells and/or for the treatment of actinic keratosis, comprising the steps:
a) measure the concentration (IC50) of a test compound, which leads to a 50% reduction in cell number in a squamous cell carcinoma (SCC) cell population showing a high-proliferation,
b) measure the concentration (IC50) of the test compound, which leads to a 50% reduction in cell number in a squamous cell carcinoma (SCC) cell population showing a low-proliferation,
c) divide the (IC50)-value from step b) by the (IC50)-value from step a) in order to get to the differential cytotoxicity factor (DCF),
d) evaluate or compare the DCF-value of the test compound with known (established) DCF-value of established drug compounds in the therapeutic field of actinic keratosis, such as 5-fluorouracil.

This method for identifying compounds can be preferably applied, in which the high-proliferation population is generated by incubation of the assay cells in culture media containing 20% fetal-bovine-serum (FBS) and the low-proliferation population by cultivation the assays cell in culture media containing 0% fetal-bovine-serum (FBS). The method is particularly useful for identifying compounds for the topical treatment of actinic keratosis.

One important problem of established therapeutics for skin treatment is their lack of specificity for the malignant-target cells and thus severe side effects due to impact on the surrounding normal cells. The resulting severe side-effects contradict their use for the desired field-treatment. A main characteristic of malignant actinic keratosis cells in comparison to the normal keratinocyte is their proliferative phenotype. In normal epidermis, only a fraction of transiently dividing cells in the basal cell layers is responsible for constantly renewing the epidermal skin layer. AK-cells escape the tight control of proliferation in supra-basal skin-layers and continue to proliferate. This effect can lead to a histological situation where focal proliferative AK-cells are surrounded by non-proliferating, normal (i.e. non-malignant) cells.

According to the method of this invention, an ectopic proliferative phenotype of AK cells was used as target for an effective and tolerable field-treatment of actinic keratosis.

In order to identify compounds with the desired differential toxicity, a suitable in-vitro test system was developed. This test system can provide cells with a high-proliferation, and a cell population with a low-proliferation phenotype. Normal in vitro cell culture systems can be optimized for maximum growth of cells in order to allow their expansion. This can be used for in vitro culture systems for normal human epidermal keratinocytes (NHEKs). Thus, although non-malignant, those culture cells would show the high-proliferative in vivo phenotype of AK cells. To generate a non- or low-proliferating cell population, it is possible to induce differentiation in the in vitro cultivated NHEKs. Although in general possible, this procedure is time-consuming and often generates in-homogenous population of cells and thus requires high efforts to reach reproducible results. Therefore, a cell culture system was chosen which is more suitable for screening purposes.

In addition to primary NHEKs there are also SCC-derived keratinocyte cell lines available (e.g. SCC-111; squamous cell carcinoma). Those SCC-cells have specific requirements to their growth-medium and often require feeder-cells or at least high-amounts of bovine-serum (e.g. 20% FBS; fetal bovine serum) as growth promoting culture supplement. It now was surprisingly found that those SCC-cell lines can be propagated in culture for several days without any serum (0%). These serum-deprived culture conditions lead to a significant reduction of the proliferation of SCC-cells. The test-system with SCC-111 cells cultivated in either 20% or 0% FBS allows the differential analysis of cytotoxic compounds based on proliferation as phenotypic characteristic.

In order to determine the differential cytotoxic effect of a compound, the IC50-value (concentration of the compound which resulted in 50% reduction of cell number) of this compound is established in 20% and in 0% FBS cultivated SCC-111 cells.

In case a compound is preferentially affecting proliferating cells the IC50-value should be higher in 0%-FBS cultivated cells (i.e. the low-proliferating cells are less sensitive to the compound and thus higher amount of compound is needed to reduce 50% of the cells). By dividing the IC50-value obtained in 0% FBS by the IC50-value obtained in 20% FBS cultivated cells, an important characteristic, called differential cytotoxicity factor (DCF) can be calculated.

In vitro SCC-cells have special requirements on culture conditions. Addition of 20% fetal-bovine-serum (FBS) is required to maintain proliferative phenotype and allow efficient seeding of cells. By switching to serum-free media (0% FBS), the proliferation of the cells can be reduced over a period of 72 hrs.

The method for identifying compounds for the targeting of proliferating epidermal cells and/or for the treatment of actinic keratosis according to this invention allows the easy screening and identification of the differential cytoxic effect of the tested compounds based on its ability to target cell-proliferation.

This differential activity is a key-success-factor for an efficient but nevertheless well-tolerated topical, field treatment of actinic-keratosis.

The screening of several known cytotoxic compound currently used only as systemic cytotoxic agents (with exception of 5-FU) demonstrates large differences concerning their specificity for proliferating cells. Furthermore the topical use for the compounds found to be particularly good was not yet described in the prior art. The described method is suitable for identifying from a large group of existing cytotoxic-compounds (as well as from groups of novel compounds) those which have a clean activity on proliferating keratinocytes, leaving non-proliferating keratinocytes rather unaffected. The therapy with such a target-cell-specific treatment results in a more efficient and tolerable treatment for the patients, in particular for actinic keratosis.

In some embodiments, the present invention provides a composition, compound, or use thereof, according to any of embodiments (a) through (w), below:
a) Pharmaceutical composition for topical application for improving the appearance of damaged skin comprising at least one vinca alkaloid or a pharmaceutically acceptable salt thereof and comprising at least one dermatologically acceptable excipient.
b) Pharmaceutical composition for the treatment of unwanted or ectopic proliferating skin-cells.
c) Pharmaceutical composition for the treatment of actinic keratosis.
d) Pharmaceutical composition, comprising a vinca alkaloid of formula (I)

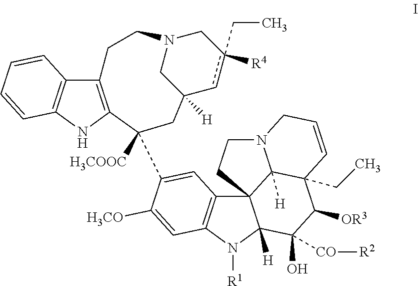

or a pharmaceutically acceptable salt (or hydrate) thereof, wherein:
R¹ is C₁-C₃-alkyl, C₁-C₃-alkoxy, or —CHO;
R² is C₁-C₃-alkyl, C₁-C₃-alkoxy, or amino;
R³ is CO—CH₃, methyl, or hydrogen,
R⁴ denotes hydroxyl or hydrogen.
e) Pharmaceutical composition, comprising a vinca alkaloid from the group consisting of vindesine, vinblastine, vincristine and vinorelbine and the pharmaceutically acceptable salts thereof
f) Pharmaceutical composition, comprising from 0.0001 to 10.0% by weight, in particular 0.001 to 10.0% by weight, such as 0.001 to 0.01%, in particular 0.002 to 0.008% by weight, such as 0.005% of the vinca alkaloid or a pharmaceutically acceptable salt thereof
g) Pharmaceutical composition for the treatment of actinic keratosis in patients, where the composition comprises 0.0001 to 10.0% by weight, in particular 0.001 to 10.0% by weight, such as 0.001 to 0.01% by weight, in particular 0.002 to 0.008% by weight, such as 0.005% by weight, of the vinca alkaloid or a pharmaceutically acceptable salt thereof, and further comprises as excipient(s) one or more component from the group of glycerol-monostearate, medium-chain fatty acid triglycerides and isopropyl myristate.
h) Pharmaceutical composition for the once daily application for the treatment of actinic keratosis, for a period of 10 to 40 days, preferably 5 to 30 days.
i) Pharmaceutical composition, wherein the composition is a cream, a lotion, a gel or a cream-gel.
l) Pharmaceutical composition for the treatment of actinic keratosis, where the composition comprises 15 to 25% by weight of ethanol.
m) Pharmaceutical composition for the treatment of actinic keratosis, where the composition has a long-term stability at a temperature of 37° C. and a pH-value below 6 of more than one month, in particular more than 3 months.
n) Process for the preparation of a pharmaceutical composition for the topical application on damaged skin comprising at least one vinca alkaloid or a pharmaceutically acceptable salt thereof and comprising at least one dermatologically acceptable excipient, comprising the step of mixing the components.
o) A kit of parts comprising a device for the separated storing and mixing of a vinca alkaloid with the excipient(s), comprising a container which has an open end and a closed end, and comprising a cap which is arranged at the open end of the container, where the container has two or more chambers which are separated from each other by a wall, wherein the wall extends from the closed end to the open end of the container, and where at least one projecting element is constructed in the cap in such a manner that the vinca alkaloid from one of the chambers can be mixed by the projecting element with the excipients of the other chamber of the container.
p) A method for identifying compounds for the targeting of proliferating epidermal cells and/or for the treatment of actinic keratosis, comprising the steps:
   a) measure the concentration (IC₅₀) of a test compound, which leads to a 50% reduction in cell number in a squamous cell carcinoma (SCC) cell population showing a high-proliferation,
   b) measure the concentration (IC₅₀) of the test compound, which leads to a 50% reduction in cell number in a squamous cell carcinoma (SCC) cell population showing a low-proliferation,
   c) divide the (IC₅₀)-value from step b) by the (IC₅₀)-value from step a) in order to get to the differential cytotoxicity factor (DCF),
   d) evaluate or compare the DCF-value of the test compound with known DCF-value of established drug compounds in the therapeutic field of actinic keratosis, such as 5-fluorouracil.
q) A method for identifying compounds, in which the high-proliferation population is generated by incubation of the assay cells in culture media containing 20% fetal-bovine-serum (FBS) and the low-proliferation population by cultivation the assays cell in culture media containing 0% fetal-bovine-serum (FBS).
r) A method for identifying compounds according to claim 14 or 15, in which compounds for the topical treatment of actinic keratosis are identified.
s) A vinca alkaloid of formula (I)

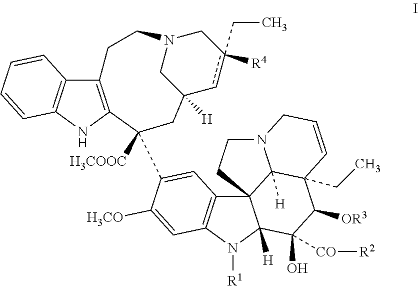

or a pharmaceutically acceptable salt (or hydrate) thereof, wherein:
R¹ is C₁-C₃-alkyl, C₁-C₃-alkoxy, or —CHO;
R² is C₁-C₃-alkyl, C₁-C₃-alkoxy, or amino;
R³ is CO—CH₃, methyl, or hydrogen; and
R⁴ is hydroxyl or hydrogen, for use in the topical treatment of sun damaged skin, in particular for use in the topical treatment of actinic keratosis.
t) The vinca alkaloid or a pharmaceutically acceptable salt thereof, wherein the vinca alkaloid is selected from the group consisting of vindesine, vinblastine, vincristine, vinorelbine and the pharmaceutically acceptable salts thereof.
u) The vinca alkaloid or a pharmaceutically acceptable salt thereof wherein the vinca alkaloids is for use in the field treatment of actinic keratosis.
v) Method for the treatment of sun damaged skin, in particular of actinic keratosis by topically administering a vinca alkaloid of formula (I)

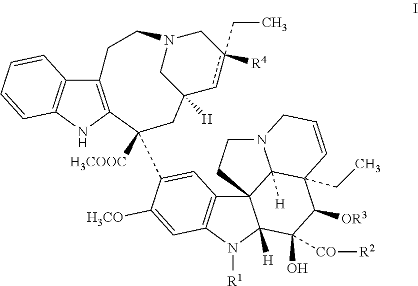

or a pharmaceutically acceptable salt (or hydrate) thereof, wherein:
R¹ is C₁-C₃-alkyl, C₁-C₃-alkoxy, or —CHO;
R² is C₁-C₃-alkyl, C₁-C₃-alkoxy, or amino;
R³ is CO—CH₃, methyl, or hydrogen; and
R⁴ is hydroxyl or hydrogen.
w) Method for the treatment of sun damaged skin, in particular of actinic keratosis, wherein the vinca alkaloid is selected from the group consisting of vindesine, vinblastine, vincristine, vinorelbine and the pharmaceutically acceptable salts thereof

The following examples and claims further illustrate the invention.

### EXEMPLIFICATION

### Example 1a

Method for identifying compounds for AK-treatment

Several known cytotoxic compounds were analyzed for the differential cytotoxicity potential. Based on calculated DCF-values (in cultivated SCC-111 cells) a ranking of the compounds could be established. The following cell-lines from DSMZ (Deutsche Sammlung von Mikroorganismen and Zellkulturen) were used:

| Supplier | Order No. | Cell-line | Species | Cell type | Origin |
|---|---|---|---|---|---|
| DSMZ | ACC666 | UPCI- | human | Squa- | Oral squamous |
| | | SCC111 | (Homo | mous cell | cell carcinoma of |
| | | | sapiens) | carcinoma | 69-year-old |
| | | | | | Caucasian woman |

The following known drug compounds were tested according to the method of the invention described:

| Test compounds | Stock concentration in DMSO | |
|---|---|---|
| 6-Thioguanine | 50 | mM |
| Bendamustine-HCl | 100 | mM |
| Doxorubicin HCL | 17 | mM |
| Epirubicin HCL | 10 | mM |
| Etoposide | 10 | mM |
| Idarubicin HCL | 10 | mM |
| Irinotecan HCL | 10 | mM |
| Melphalan | 89.13 | mM |
| Oxaliplatin | 10 | mM |
| Paclitaxel | 10 | mM |
| Teniposide | 10 | mM |
| Vinblastine sulfate hydrate | 10 | mM |
| Vincristine sulfate | 1.083 | mM |
| Vindesine sulfate hydrate | 10 | mM |
| Vinorelbine | 10 | mM |
| 5-Fluorouracil | 384.3 | mM |

For the testing, dimethylsulfoxid was used as solvent for the drugs (Supplier: AppliChem, Product No. A3009.0250 Dimethylsulfoxide pure, Ph.Eur). Further, PBS (phosphate buffered saline) was used as solvent for detection kit and for protection of evaporation in cell culture plates: PBS Dulbecco w/o Ca²⁺, w/o Mg²⁺, low endotoxin (Cat. No. L1820 Biochrom).

The compositions of the established media were:

| SCC111- | | | |
|---|---|---|---|
| Media | Components | | |
| 0% FBS | 500 ml MEM (1x) (+)Earl's | 0 ml FBS | 5 ml Glutamax |
| Medium | salts, +NEAA , −L-Glutamine | | (GlutaMaX ™ 100x, |
| | (10370-047/Gibco Invitrogen) | | 35050/Gibco Invitrogen) |
| 20% FBS | 500 ml MEM (1x) + Earl's salts, | 100 ml FBS | 6 ml Glutamax |
| Medium | +NEAA, −L-Glutamine (10370- | (FBS-Superior | (GlutaMaX ™ 100x, |
| | 047/Gibco Invitrogen) | standardized; | 35050/Gibco Invitrogen) |
| | | S0615/Biochrom) | |
| 40% FBS | 500 ml MEM (1x) + Earl's | 200 ml FBS | 7 ml Glutamax |
| Medium | salts, +NEAA, −L-Glutamine | (FBS-Superior | (GlutaMaX ™ 100x, |
| | (10370 047/Gibco Invitrogen) | standardized; | 35050/Gibco Invitrogen) |
| | | S0615/Biochrom) | |

The complete formulation of MEM medium is described on the product site of Invitrogen homepage (quoting product no. 10370-047).

As common detection kit, CyQuant® Direct cell proliferation assay kit (Cat. No. C35012 Life Technologies) was used.

The following culture flask and culture plates were used:
flask: 175 cm² Nunclon Delta treated flask, blue filter cap
   (Cat. No. 159910 Thermo Scientific Nunc)
culture plate: black 96 well plate with clear bottom
   (Cat. No. 165305 Thermo Scientific Nunc)
transparent 96 well plate (Cat. No. 161093 Thermo Scientific Nunc).

For the Cultivation of Squamous cell carcinoma 111 (SCC111) the following method was used:

SCC111 cells were cultured in MEM containing 20% FBS medium at 37° C. and 5% CO2 to 70-80% confluency. For the experiments, cells were seeded at 12500 cells/cm2 into black 96-well optical flat-bottom tissue culture plates. The cells were incubated overnight to allow attachment. In order to generate a population showing reduced proliferation (low-proliferation cells) the media was replaced by MEM containing 0% FBS. For generation of the cell population showing the maximum cell proliferation (high-proliferating cells) the cells are further cultivated in MEM containing 20% FBS.

The test compounds (see table of compounds above) were prepared according to Table 1 to yield respective stock solutions. Said stock solutions were further diluted in assay media to obtain different test compound concentrations.

The final concentrations in cell culture were reached by 1000-fold dilution of the respective DMSO-stock in the culture media.

TABLE 1
Test item solutions (example)

| Stock | | | | | | |
|---|---|---|---|---|---|---|
| solution | | | Serial stock solutions | | Final in culture | |
| No. | Substance | Solvent | (100% DMSO) | | (0.1% DMSO) | |
| 1 | 5-FU | DMSO | 100 | mM | 100 | μM |
| 2 | | | 10 | mM | 10 | μM |
| 3 | | | 1 | mM | 1 | μM |
| 4 | | | 0.1 | mM | 100 | nM |
| 5 | | | 10 | μM | 10 | nM |
| 6 | | | 1 | μM | 1 | nM |
| 7 | | | 0.1 | μM | 0.1 | nM |
| 8 | | | 10 | nM | 0.01 | nM |

The different 1000-fold-stock-solutions are generated by serial dilution starting with stock solution no. 1. One part of the respective stock solution is mixed with 500 parts 0% FBS medium according to table Dilution step 1, following the stock-solutions underwent a final 1:2 dilution by addition to the cell culture media placed in the plates (see table dilution step 2). In case of high proliferating cells, wells are prefilled with 100 μl of 40% FBS medium. For low proliferating cells, wells are prefilled with 100 μl 0FBS medium.

By final 1:2 dilution of test item in 0% FBS medium with 40% FBS medium in prefilled wells the final concentration of test item in 20% FBS is realized for the high proliferating cells.

Dilution step 1:

| | | | Volume | | | |
|---|---|---|---|---|---|---|
| | | Volume of | of 0% | | | |
| Stock solution | Stock | stock | FBS- | Total | Dilutional | Concentration |
| (100% DMSO) | concentration | concentration | medium | volume | factor | in tube |
| Stock solution | 100 mM | 5 μl | 2.495 ml | 2500 | 500 | 0.2 mM |
| No. 1 | | | | | | |

Dilution step 2:

| | | | Volume | | | |
|---|---|---|---|---|---|---|
| | | | of 0% | | | |
| | | | or 40% | | | |
| | | | FBS- | Total | | |
| | | | medium | volume | Dilutional | Concentration |
| New Stock | | | in 96 | in 96 | factor | in |
| solution | | Volume of | cell- | cell- | in 96 cell- | 96 cell- |
| (0.2% DMSO) | Stock | new stock | culture- | culture- | culture- | culture- |
| in tube | concentration | concentration | well | well | well | well |
| 1:500 Stock | 0.2 mM | 100 μl | 100 μl | 200 μl | 2 | 0.1 mM |
| solution in 0% | | | | | | |
| FBS medium | | | | | | |

In order to minimize variations in dilutions for high and low proliferating cells, one masterplate for both cell populations was prepared for the transfer of the serial 500-fold diluted stock solutions. The masterplate contained the serial dilution solution of a test compound for high and low proliferating cells and media for the negative controls. Where possible, the screenings were carried out with a maximal solvent concentration of 0.1% (v/v); in exceptional cases a maximum of 0.2% DMSO was used. The negative control wells contained media only, plus the respective solvent. Unused portions of the stock solutions were stored at −20° C.

The following experimental setup was used for determination of cytotoxic potential of test compounds:

The previous day, SCC111 cells were seeded in 20% FBS-media at 12500 cells/cm2 to allow attachment overnight. When attached, medium was aspirated and replaced by 100 μl 0% FBS medium or by 100 μl 40% FBS medium. Afterwards 100 μl of 500-fold diluted serial stock solution of the test compound was added.

4 replicates of each test compound concentration were tested. Each plate included a negative control (4 replicates) and a compound interference control (for all concentrations, triplicates).

The amount of DNA, as surrogate for cell number, was determined 72 hours after incubation with the test compound. Using a commercial plate reader (SynergyH4), the CyQuant Dye, that produces a fluorescence signal after DNA intercalation, was recorded (at 480Ex/535Emnm).

The following protocol for determination of cell numbers using the commercial CyQuant-assay was used:

CyQuant® kit components:
A) CyQuant Direct nucleic acid stain
B) CyQuant Direct background suppressor I

The two CyQuant-components were mixed together with PBS and added to the culture media according to protocol provided by the supplier (see Life Technologies, Cat. No. C35012). The following recipe is for preparing 12 mL of 2-fold concentrated detection reagent, which is sufficient for one microtiter plate with 100 μL/well in a 96-well plate:

| | | |
|---|---|---|
| PBS | 11.7 | mL |
| CyQuant ® Direct nucleic acid stain | 48 | μL |
| CyQuant ® Direct background suppressor I | 240 | μL. |

Add 100 μL of 2-fold concentrated detection reagent to 100 μL of cells in cell culture media. Incubate cells with the detection reagent for 60 minutes at 37° C. Read fluorescence of samples using appropriate wavelengths (480/535 nm, monochromator-based instrument).

The following data analysis of cell number (proliferation) was used:

Mean values of relative fluorescence units (RFU) and standard deviation of replicates were analysed by Gen5 software.

To create a sigmoidal IC50 curve, cell number (proliferation) measured in untreated control cells were set as 100%. Using the commercial SigmaPlot Version12.3, sigmoidal curves were generated from which the relative IC-50 values are determined.

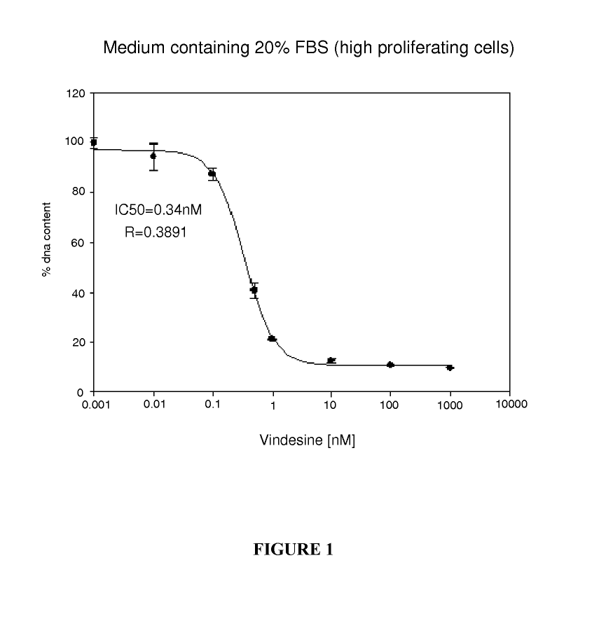

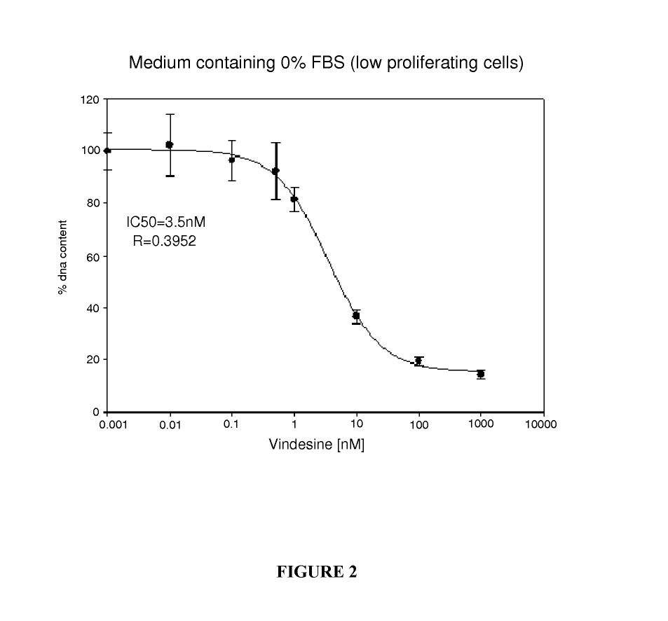

The differential cytotoxicity factor (DCF) was established by dividing the obtained IC50-value in 0% FBS medium by the IC50-value obtained in 20% FBS medium cultivated cells. An example of this method applied for the vinca-derivative Vindesine is given in the enclosed FIGS. 1 and 2, where the DNA-content (%) is shown as a function of Vindesine concentration (nM) for 20% FBS medium cultivated cells (FIG. 1) and for 0% FBS medium cultivated cells (FIG. 2). From these curves, for FIG. 1 an IC50-value was found at 0.34 nM, for FIG. 2 an IC50-value was found at 3.5 nM, leading to DCF-value >10.

The DCF-value (differential cytotoxicity factor), defining how much-fold proliferating cells are more affected in 20% FBS (High proliferation) than in 0% FBS (low proliferation) was found to be a good indicator for a compounds activity for actinic keratosis activity. Considering these factors of the various known compounds tested, the following four vinca-alkaloids performed particularly well.

After repeating the experiments several times, the following ranking was found:

| Compound | DCF-value |
|---|---|
| Vindesine | 11.43 |
| Vinblastine | 19.87 |
| Vincristine | 5.86 |
| Vinorelbine | 6.42 |

Vinblastine shows the highest DCF-value, vinorelbine is of particular interest for its particular formulation advantages.

Also of some interest were the drug compounds Epirubicin (DCF of 6.05) and Teniposide (DCF of 5.76), which however are no vinca alkaloids and therefore do not have some of the advantages of these alkaloids. For the known drug compound 5-FU, DCF-value of 0.73 was measured.

### Example 1b

### Dermal Absorption Study with Vinca Alkaloids

Skin Preparations: Female human abdominal skins from plastic surgery were used. The subcutaneous fatty tissue was removed from the skin and then frozen at −20° C. with aluminium foil packed for later tests. Before use, skin disks of 18 mm diameter were punched, cleaned with PBS (phosphate buffered saline, pH 7.4, 10 mM) and thaw with the stratum corneum (SC) side up open to the atmosphere and the dermal side bathed with PBS for 2 hrs. The thickness of the skin was estimated to exhibit an average of 2 mm.

Penetration Study: The skin samples were mounted in Franz diffusion cells under non-occlusive condition with the effective surface area of 0.5 cm2 and a receptor volume of 5.1 ml. The dermal side of the skin was exposed to the receptor fluid (PBS) and the SC remained in contact with the donor compartment left dry and opened to the atmosphere. The temperature of the water bath was maintained at 37±0.1° C. The receptor solution was continuously stirred at 700 rpm with a small bar magnet placed inside the cell. Ten μl of Hydro-Tops or liposomes or 10 mg of the cream formulation were applied onto the skin's surface and uniformly spread by pipettes or spatula. The spatula was weighted again after the application of the cream to determine the applied dose. This means an application dose of 1 μg API per square cm. Samples (0.7 ml) were taken from the receptor fluid at 3, 6, 12, 18 and 24 hrs after application. At each point, the receptor cell content was replaced by 0.7 ml of fresh PBS solution. Three replicates were used for the studies (same skin preparations)

Tape Stripping: The distribution of API in the stratum corneum was determined by the tape stripping technique. After 24 hours the skin was taken, stretched and mounted with pins on cork discs and covered with aluminium mask with a central hole of 8 mm in diameter. The SC was successively removed by stripping with an adhesive tape (Cristall KlarTesa®, Beiersdorf). Twenty stripping procedures were performed consecutively. The first five strips were separately transferred and strips 6-10, 11-15 and 16-20 were pooled and transferred into vials of suitable size. To elute the 3H-API from the strips, 2 ml of ethanol/PBS pH 7.4 (1:1, v/v) was added. The samples were vortexed for 2 min and sonicated for 30 min in a water bath at room temperature.

Measurement of 3H-API: The samples (standards, strips and fluids) were mixed with 12 ml Rotiszint eco plus (scintillation cocktail purchased from Carl Roth) and carefully vortexed for at least 1 minute. The skin is divided into epidermis and dermis by a scalpel and solubilized in 1 ml Soluene 350 (Perkin Elmer). The samples were shaken at 55° C. for 6 hours to get a clear solution and then mixed with 12 ml Hionic Fluor (Perkin Elmer). The activity in each sample was determined in a scintillation counter (Tri-Carb 2910TR Liquid Scintillation Analyzer; Perkin Elmer) by a standardized protocol.

For vinorelbine ditartrate as API in a crème formulation (Example 11) and in a hydro-tops formulation (Example 5), penetration values of >0.7% in the epidermis were found.

### Example 2

### Formulation of Vindesine (Crème) for AK-Treatment

Based on the data found for the various vinca alkaloids, a topical field treatment using suitable semi-solid formulations with these vinca alkaloids is tested.

In particular, the following topical formulation of Vindesine is proposed.

A composition (basic cream) of the following ingredients is prepared (in g):

| | |
|---|---|
| Vindesine | 1.5 |
| Glycerol-monostearate (60) | 4.5 |
| Cetyl alcohol | 6.0 |
| Miglyol 812 | 7.5 |
| Vaseline (white) | 25.5 |
| Propylene-glycol | 10.0 |
| Macrogol-20-glycerol-monostearate | 7.0 |
| Water | 38.0 |

This composition can easily be administered at the skin and is useful for the once daily treatment of actinic keratosis.

### Example 3

### Treatment of Actinic Keratosis

A topical vindesine formulation (cream) can be used for the treatment of actinic keratosis. On the skin either 5 drops of placebo (same composition, but without vinca alkaloid) or 5 drops of the topical vindesine cream formulation (e.g. from example 2) are placed on waterproof occlusion bandages (6.5 cm×5 cm). Subsequently, the occlusion bandages can be placed on the left arm (Vindesine) and the right arm (placebo) of a subject suffering from AK. The occlusion bandages can be renewed once daily. When the bandages are renewed, remains of old administered substances can be removed, before placing a fresh, occlusion bandage with vinca or Placebo.

### Example 4

### Device for Vinca Alkaloid and Excipients for AK-Treatment

A device for the separated storing and mixing of a vinca alkaloid with the excipient(s) can be used. As example, the device shown in WO 2012/069441 can be used. This device can be a two-chamber primary-packing, in which the freeze-dried vinca alkaloid (Vindesine) is kept separately from the base formulation containing the excipients (see example 2). Prior to use by the patient, the two compartments are combined and mixed in order to get to the topical vinca formulation which then is ready for use. As one further variant a 3-chamber system is proposed, in which the vinca alkaloid is dissolved in a solvent of low viscosity first, before combining this mixture with the semi-solid components of the final vinca formulation.

### Example 5

### Vinorelbine Formulation for AK-Treatment

The topical Vinorelbine ditartrate formulation (hydro-tops) is prepared by using the following ingredients (in g):

| | |
|---|---|
| Vinorelbine ditartrate | 0.005 |
| Glyceryl Citrate, Lactate, | 7.0 |
| Linoleate, Oleate (Imwitor375) | |
| Ethanol | 20.0 |
| Ethyl Oleate (Crodamol-EO) | 3.0 |
| Water | 69.995 |

The following process steps were applied:
(a) Imwitor® (emulsifier) and Crodamol-EO are solubilized in ethanol
(b) Vinorelbine ditartrate is solubilized in water.

Vinorelbine ditartrate has a solubility of 0.7% at pH 6.5.

The vinca/water solution is added to the emulsifier solution under homogenization (Ultra-Turrax® 20.000 U/min). Additional homogenization for 5 minutes with 20.000 U/min is applied. High pressure homogenization follows with 1-3 cycles at 800 bars. This composition can easily be administered at the skin and is useful for the once daily treatment of actinic keratosis.

### Example 6

### Vinblastine Formulation for AK-Treatment

The topical Vinblastine sulfate formulation (hydro-tops) is prepared by using the following ingredients (in g):

| | |
|---|---|
| Vinblastine sulfate | 0.005 |
| Glyceryl Citrate, Lactate, | 7.0 |
| Linoleate, Oleate (Imwitor375) | |
| Ethanol | 20.0 |
| Ethyl Oleate (Crodamol-EO) | 3.0 |
| Water | 69.995 |

The following process steps were applied: Vinblastine sulfate has a solubility of 0.25% at pH 6.5.
Imwitor and Crodamol are solubilized in ethanol
Vinblastine sulfate is solubilized in water.

The vinca/water solution is added to the emulsifier solution under homogenization (Ultra-Turrax® 20.000 U/min). Additional homogenization for 5 minutes with 20.000 U/min. High pressure homogenization 1-3 cycles at 800 bars. This composition can easily be administered at the skin and is useful for the once daily treatment of actinic keratosis.

### Example 7

### Teniposide Formulation for AK-Treatment

A topical Teniposide formulation (hydro-tops) is prepared by using the same ingredients as in example 6 but using 0.005 g of teniposide as active ingredient. This composition can easily be administered at the skin and is useful for the once daily treatment of actinic keratosis.

### Example 8

### Vinorelbine Liposome Formulation for AK-Treatment

The topical vinorelbine ditartrate formulation (liposomes) is prepared by using the following ingredients (in g):

| | |
|---|---|
| Vinorelbine ditartrate | 0.005 |
| Phospholipon 90G (PC) | 10.0 |
| Ethanol | 20.0 |
| Water (and phosphate buffer) | 69.995 |

The following process steps were applied:

Phopholipids (phosphatidyl choline) are solubilized in ethanol till clear solution is obtained, Vinorelbine ditartrate is solubilized in water. Vinca alkaloid and water solution is added to the lipid solution under homogenization (Ultra-Turrax® 20.000 U/min). Additional homogenization occurs for 5 minutes with 20.000 U/min. High pressure homogenization is made by 1-3 cycles at 800 bars. This composition can easily be administered at the skin and is useful for the once daily treatment of actinic keratosis.

### Example 9

### Vinblastine Liposome Formulation for AK-Treatment

The topical Vinblastine sulfate formulation (liposomes) is prepared by using the following ingredients (in g):

| | |
|---|---|
| Vinblastine sulfate | 0.005 |
| Phospholipon 90G (phosphatidyl choline) | 10.0 |
| Ethanol | 20.0 |
| Water (and phosphate buffer) | 69.995 |

The following process steps were applied: Phopholipids are solubilized in ethanol till clear solution is obtained, Vinorelbine is solubilized in water. Vinca alkaloid and water solution is added to the lipid solution under homogenization (Ultra-Turrax® 20.000 U/min). Additional homogenization is used for 5 minutes with 20.000 U/min. High pressure homogenization 1-3 cycles at 800 bars. This composition can be administered at the skin and is useful for the once daily treatment of actinic keratosis.

### Example 10

### Teniposide Liposome Formulation for AK-Treatment

A topical formulation (liposomes) with teniposide is prepared by using the same ingredients as in example 9 but using 0.005 g of teniposide as active ingredient.

The following process steps were applied: Teniposide is solubilized in ethanol till clear solution is obtained, Phospholipis are solubilized in Teniposide/ethanol.

Water is added to the lipid solution/Teniposide solution under homogenization (Ultra-Turrax® 20.000 U/min). Additional homogenization for 5 minutes with 20.000 U/min. High pressure homogenization 1-3 cycles at 800 bars. This composition can easily be administered at the skin and is useful for the once daily treatment of actinic keratosis.

### Example 11

### Vinorelbine Cream Formulation for AK-Treatment

A composition (basic cream) of vinorelbine ditartrate is prepared based on the ingredients (in g) of the following basis cream:

| | |
|---|---|
| Polysorbate (60) | 4.0 |
| Cetyl alcohol | 6.0 |
| Caprylic/capric-triglycerides | 7.5 |
| Petrolatum | 25.5 |
| Propylene glycol | 10.0 |
| PEG-20 Glycerol-stearate | 7.0 |
| Water | 40.0 |

Then, the vinca compound Vinorelbine ditartrate (0.005 g), ethanol (20 g), water (10 g) and the basic cream (69.995 g) were mixed. This composition can easily be administered at the skin and is useful for the once daily treatment of actinic keratosis.

### Example 12

### Vinblastine Cream Formulation for AK-Treatment

A composition (basic cream) of vinblastine sulfate is prepared based on the ingredients (in g) of the following basis cream:

| | |
|---|---|
| Polysorbate (60) | 4.0 |
| Cetyl alcohol | 6.0 |
| Caprylic/capric-triglycerides | 7.5 |
| Petrolatum | 25.5 |
| Propylene glycol | 10.0 |
| PEG-20 Glycerol-stearate | 7.0 |
| Water | 40.0 |

Then, the vinca compound vinblastine sulfate (0.005 g), ethanol (20 g), water (10 g) and the basic cream (69.995 g) were mixed. This composition can easily be administered at the skin and is useful for the once daily treatment of actinic keratosis.

### Example 13

### Vincristine Cream Formulation for AK-Treatment

In analogy to example 12, a crème is prepared containing 0.01% by weight of vincristine sulfate (instead of Vinblastine sulfate). The solubility of vincristine sulfate in water at pH 5.5 was 0.57% by weight

We claim:

1. A pharmaceutical composition for topical administration to a patient in need thereof, comprising at least one vinca alkaloid or a pharmaceutically acceptable salt thereof and comprising at least one dermatologically acceptable excipient.

2. The composition according to claim 1, wherein said patient has unwanted or ectopic proliferating skin-cells.

3. The composition according to claim 2, wherein said patient has actinic keratosis.

4. The composition according to claim 1, wherein the vinca alkaloid is of formula (I): 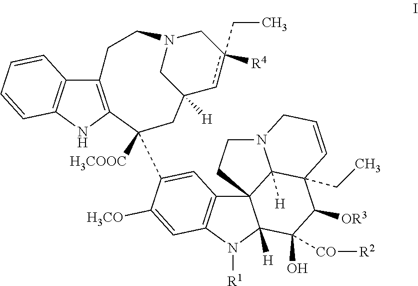
or a pharmaceutically acceptable salt (or hydrate) thereof, wherein:
R¹ is C₁-C₃-alkyl, C₁-C₃-alkoxy, or —CHO;
R² is C₁-C₃-alkyl, C₁-C₃-alkoxy, or amino;
R³ is CO—CH₃, methyl, or hydrogen; and
R⁴ is hydroxyl or hydrogen.

5. The composition according to claim 4, wherein:
R¹ is methyl or CHO;
R² is methoxy or amino; and
R⁴ is hydroxyl.

6. The composition according to claim 1, wherein the vinca alkaloid is selected from vindesine, vinblastine, vincristine and vinorelbine, or a pharmaceutically acceptable salt thereof.

7. The composition according to claim 3, wherein said composition comprises from 0.0001 to 10.0% by weight of the vinca alkaloid, or a pharmaceutically acceptable salt thereof.

8. The composition according to claim 7, further comprising an excipient selected from glycerol-monostearate, medium-chain fatty acid triglycerides or isopropyl myristate.

9. The composition according to claim 8, wherein said composition is administered to said patient once daily for 10 to 40 days.

10. The composition according to claim 1, wherein said composition is a cream, a lotion, a gel, or a cream-gel.

11. The composition according to claim 3, further comprising 15 to 25% by weight of ethanol.

12. The composition according to claim 1, wherein said composition is stable at a temperature of 37° C. and a pH-value below 6 for more than one month.

13. A method of treating actinic keratosis in a patient in need thereof, wherein said method comprises topically administering to said patient the composition according to claim 1.

14. The method according to claim 13, wherein the vinca alkaloid is selected from vindesine, vinblastine, vincristine, vinorelbine or a pharmaceutically acceptable salt thereof.

15. The method according to claim 13, wherein the vinca alkaloid is of formula (I): 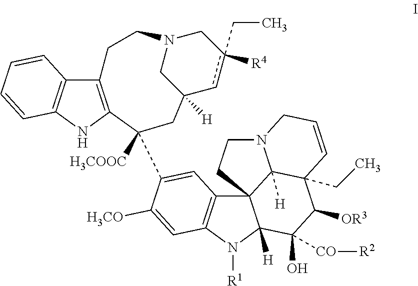
or a pharmaceutically acceptable salt (or hydrate) thereof, wherein:
R¹ is C₁-C₃-alkyl, C₁-C₃-alkoxy, or —CHO;
R² is C₁-C₃-alkyl, C₁-C₃-alkoxy, or amino;
R³ is CO—CH₃, methyl, or hydrogen; and
R⁴ is hydroxyl or hydrogen.

16. The method according to claim 13, wherein.

17. The method according to claim 15, wherein:
R¹ is methyl or CHO;
R² is methoxy or amino; and
R⁴ is hydroxyl.

18. The composition according to claim 13, wherein said composition comprises from 0.0001 to 10.0% by weight of the vinca alkaloid, or a pharmaceutically acceptable salt thereof.

19. The composition according to claim 18, further comprising an excipient selected from glycerol-monostearate, medium-chain fatty acid triglycerides or isopropyl myristate.

20. The composition according to claim 18, wherein said composition is administered to said patient once daily for 10 to 40 days.
